(19) United States
(12) Patent Application Publication
(10) Pub. No.: US 2003/0032976 A1
(43) Pub. Date: Feb. 13, 2003

(54) Catheter deployed partial occlusion devices and methods

(76) Inventor: Mark M. Boucek, Golden, CO (US)

Correspondence Address: Alison B. Mohr, Parsons Behle & Latimer, 201 South Main Street, Suite 1800, Salt Lake City, UT, 84111-2218, US

(21) Appl. No.: 10/152,097

(22) Filed: May 21, 2002

Related U.S. Application Data
(60) Provisional application No. 60/292,357, filed on May 21, 2001.

(51) Int. Cl. A61M 29/00

(52) U.S. Cl. 606/200; 623/1.18

(57) ABSTRACT

A device and method of use for a partial occlusion device (POD) for matter and liquid flow control. More specifically, a device relating to the partial occlusion of blood vessels to affect the flow of blood through the blood vessels is disclosed. The POD may be formed from a mesh material and includes one or more lumens. The POD may be particularly useful in treating Hypoplastic Left Heart Syndrome, by reducing pulmonary blood flow to decrease the risk of pulmonary hypertension. The POD may also be used to deliver therapeutic agents to a specific site. The POD can be retrieved after placement.

### CROSS REFERENCE TO RELATED APPLICATIONS

This application is based on Provisional Application Serial No. 60/292,357, filed May 21, 2001, and priority is claimed thereto.

### BACKGROUND OF THE INVENTION

1. The Field of the Invention

The present inventions relate to matter and liquid flow control devices. More specifically, the present inventions relate to the partial occlusion of blood vessels to affect the flow of blood through the blood vessels.

2. The Relevant Technology

Some medical conditions require restriction of blood flow or controlled passage of an agent (for example a drug, a biochemical, or a gene therapy delivery device or virus) through a blood vessel.

For example, Hypoplastic Left Heart Syndrome (HLHS) describes several congenital abnormalities of the heart. The primary abnormality is aortic atresia (an absence or closure of the aorta) often associated with mitral atresia (an absence or closure of the orifice of the mitral valve) and subsequent hypoplasia (failure to develop) of the left ventricular cavity and ascending aorta. Collectively these malformations may be referred to as left-sided lesions. The left side of the heart indicates the left atrium, left ventricle and associated structures in and around the left atrium and ventricle. The right side of the heart indicates the right atrium, right ventricle and associated structures in and around the right atrium and right ventricle. HLHS occurs within the first trimester of pregnancy and, while not fatal during pregnancy, this syndrome is uniformly fatal shortly after birth without intervention.

Fetal circulation is different than post-natal circulation. Fetal blood is oxygenated in the placenta and returned to the heart from the placenta at the right atrium. Instead of being pumped into the right ventricle, a portion of the oxygenated blood crosses into the left atrium through the foramen ovale, bypassing the lungs. The foramen ovale is a connection between the right and left atriums that closes shortly after birth. The oxygenated blood in the left atrium passes into the left ventricle and then to the upper body through the aorta. Blood returning from the upper body is only partially deoxygenated, and is used for further tissue oxygenation. The portion of the partially deoxygenated blood that passes from the right atrium to the right ventricle is pumped out of the heart through the pulmonary artery. Rather than flowing to the lungs, a portion of this blood is bypassed to the aorta through the ductus arteriosus (also called the pulmonary ductus arteriosus). The ductus arteriosus is a connection between the pulmonary artery and the aorta and is positioned such that blood passing through the ductus arteriosus can be directed to the lower body. Deoxygenated blood from the lower body is returned to the placenta for oxygenation, and the newly oxygenated blood repeats the cycle by returning to the heart at the right atrium. Normally, the ductus arteriosus closes after birth.

In most instances, infants with HLHS must have a cardiac (heart) transplantation or complex surgical palliation. Oxygenated blood will not circulate well throughout the body of infants with left sided lesions, and without a heart transplant the infant will likely die. In the past 15 years several surgical procedures have significantly changed the prognosis for infants born with HLHS. Infant heart transplantation has resulted in five-year survival that now approximates 70%. While waiting for cardiac transplantation, infants born with HLHS can be stabilized and maintained on prostaglandin E1 infusion to maintain patency of the ductus arteriosus. The maintenance of the ductus arteriosus allows systemic perfusion (flow of oxygenated blood to the body) from the right ventricle. Partially oxygenated blood flowing from the right ventricle can pass into the aorta, bypassing the left-sided obstructive lesions of HLHS. Furthermore, an atrial communication can be created or enlarged to decompress the left-sided obstructive lesions. In essence, maintaining the foramen ovale.

However, maintaining the ductus arteriosus exposes the pulmonary bed to systemic pressures, causing pulmonary hypertension. With time pulmonary hypertension becomes progressive and irreversible making transplantation impossible generally beyond four to six months of age. At present only about three quarters of the infants listed for transplantation will receive a donor within the acceptable time frame. The longer the child waits, the higher the risk at transplantation. Hence, there is significant cost and morbidity associated with chronic prostaglandin E1 infusion and waiting for transplantation.

Another approach, the Norwood procedure, offers a surgical palliative (abating) alternative to transplantation using a three stage approach. The five-year survival for the Norwood procedure is approximately 60% with much of the mortality occurring with the initial Stage I (creation of a single artery from the right ventricle of the heart to the aorta). Following the Norwood procedure infants may still require transplantation. Thus, the current surgical approaches to management of children with HLHS are associated with significant mortality. The Norwood procedure has vastly improved survival, and many children with HLHS now have a reasonable prognosis with the formally lethal diagnosis. However, largely due to the high early risk associated with surgery, some families choose the option of no intervention, and the child dies shortly after birth.

### SUMMARY AND OBJECTS OF THE INVENTION

The inventions relate to a non-surgical, catheter-deployed palliative procedure for treating infants suffering from HLHS and awaiting transplantation that may consist of three steps. Two steps create a single ventricle physiology and are done during a single catheterization procedure. The first step is maintaining ductal patency of the ductus arteriosus by transcatheter stenting. Catheter placement of a stent in the ductus arteriosus has been successfully performed in infants. The second step is creation of an atrial communication between right and left atrium. This has been utilized in children for approximately eight-years and yields less than a five-percent mortality. The third step is performed through a second catheterization procedure to limit pulmonary blood flow to decrease the risk of pulmonary hypertension. Previous attempts to do this surgically have been problematic due to the difficulty of selectively narrowing both right and left pulmonary arteries.

The present inventions relate to the partial occlusion of blood vessels to limit blood flow or blood pressure. A preferred embodiment of the inventions limits blood flow or blood pressure through the pulmonary artery(ies) as treatment for HLHS. Partial Occlusion Devices (PODs) are devices that can be placed within a blood vessel to limit the flow and pressure of blood through the device. The ductus arteriosus must be stented open to treat HLHS. However, this subjects the pulmonary bed to systemic pressure. When PODs are placed in the pulmonary arteries during treatment of HLHS they limit the flow of blood to the lungs and thereby reduce the pressure placed on the pulmonary bed. The present inventions may be practiced by placement and removal of PODs through the use of a catheter or other similar methods that are less invasive than full surgery to access the site. However, placement and removal of PODs directly, at the time of surgery, is also envisioned.

The present inventions also relate to the restriction of blood flow and pressure, at any point within a patient (whether human or non-human) where such a restriction is needed.

In another embodiment, the present inventions relate to the restriction of the flow of matter (such as blood) through matter transport pathways (such as arteries or veins) where accessing the point where a restriction is needed is traditionally difficult or impossible. The POD can be inserted into the matter transport pathway at a point in the matter transport pathway that is accessible and then fed through the matter transport pathway utilizing a catheter or similar device. Once the POD is at the point where a restriction is needed the POD may be released from the catheter or similar device to place the POD at the desired point. The POD can be designed to fit the point where a restriction is needed, and also to affect the flow of matter in the manner needed.

It is therefore an object of the preferred embodiment of the present inventions to decrease the flow of blood through the pulmonary arteries while treating HLHS to decrease the pressure on the pulmonary bed and increase the survival rate of infants born with HLHS. It is also an object of another embodiment of the present inventions to decrease the flow of blood through any blood vessel. It is also an object of another embodiment of the present inventions to regulate and deliver agents or cells through blood vessels. It is also an object of embodiments of the present inventions to regulate the flow of matter through any matter transport pathway. The structures and methods disclosed herein may have many other uses in medical treatments and may achieve objects other or different then those stated herein.

These and other objects, features and advantages of embodiments of the present inventions will become more apparent from the following description and claims, or may be learned by the practice of the inventions as set forth hereinafter.

### BRIEF DESCRIPTION OF THE DRAWINGS

The accompanying drawings incorporated in and forming a part of the specification illustrate preferred embodiments of the present inventions. Some, although not all, alternative embodiments are described in the following description and therefore the drawings are not intended to limit the scope of the inventions. The inventions will be described and explained with additional specificity and detail through the use of the accompanying drawings where:

FIG. 1 illustrates a proximal view of a POD with a triangular lumen and an eccentrically located catheter attachment port.

FIG. 2 illustrates a proximal view of a POD with a central catheter attachment port and two lumens.

FIG. 3 illustrates a proximal view of a POD with a circular lumen and a slightly eccentric catheter attachment port.

FIG. 4 illustrates a cross-sectional side view of a tapered POD.

FIG. 5 illustrates a cross-sectional side view of a cylindrical POD.

FIG. 6 illustrates a cross-sectional side view of a cylindrical POD with lips.

FIG. 7A illustrates a cross-sectional side view of the internal configuration of an inverted funnel POD.

FIG. 7B illustrates a cross-sectional side view of the internal configuration of a funnel POD.

FIG. 7C illustrates a cross-sectional side view of the internal configuration of a cylinder POD.

FIG. 7D illustrates a cross-sectional side view of the internal configuration of a central constriction POD.

FIG. 8A demonstrates the threading of a POD upon a catheter to the desired position in the left pulmonary artery.

FIG. 8B demonstrates the release of a POD from a catheter.

FIG. 8C demonstrates the withdrawal of a catheter from the placement site of a POD.

FIG. 8D illustrates the placement of PODs in the right and left pulmonary arteries and a stent within the pulmonary ductus arteriosus to treat HLHS.

FIG. 9 illustrates the placement of a POD within the hepatic artery to treat hepatic disorders.

FIG. 10 illustrates a close up view of a catheter attachment port.

FIG. 11 demonstrates a retrieval device engaging the catheter attachment port.

FIG. 12 illustrates an alternative retrieval device.

### DETAILED DESCRIPTIONS OF EMBODIMENTS

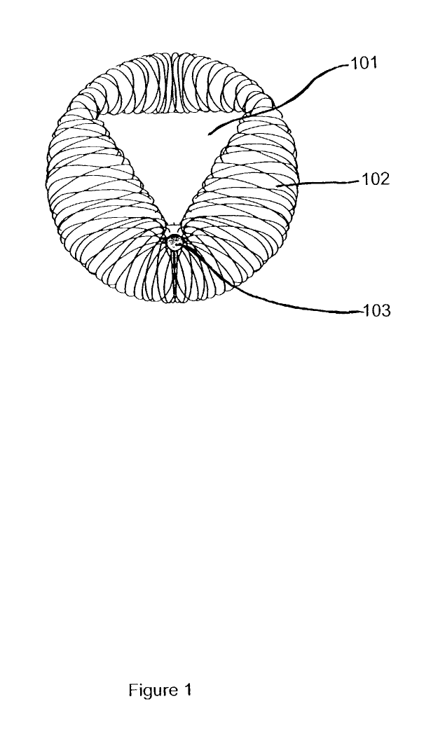

FIG. 1 illustrates a proximal view of a POD that has a triangular-shaped (in cross-section) lumen 101. The mesh 102 that makes up the occlusion device and the catheter attachment port 103 are also shown in FIG. 1. The lumen would permit flow of matter such as blood there through while the mesh would impede matter flow, thereby partially occluding a matter transport pathway.

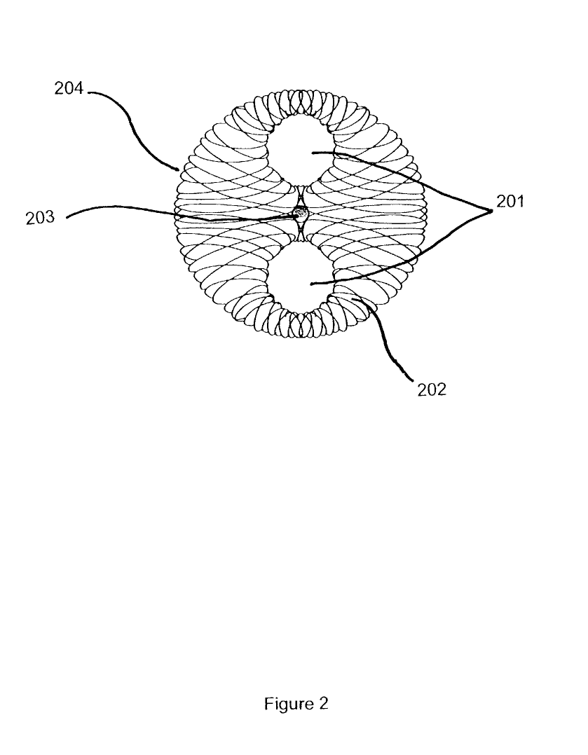

FIG. 2 illustrates a proximal view of a POD with two circular lumens 201 located eccentrically (meaning located elsewhere than the geometric center). The outer circumference of the POD 204 is roughly circular. The mesh 202 inhabits the space from the lumens 201 to the outer circumference of the POD 204. The catheter attachment port 203 is generally centrally located.

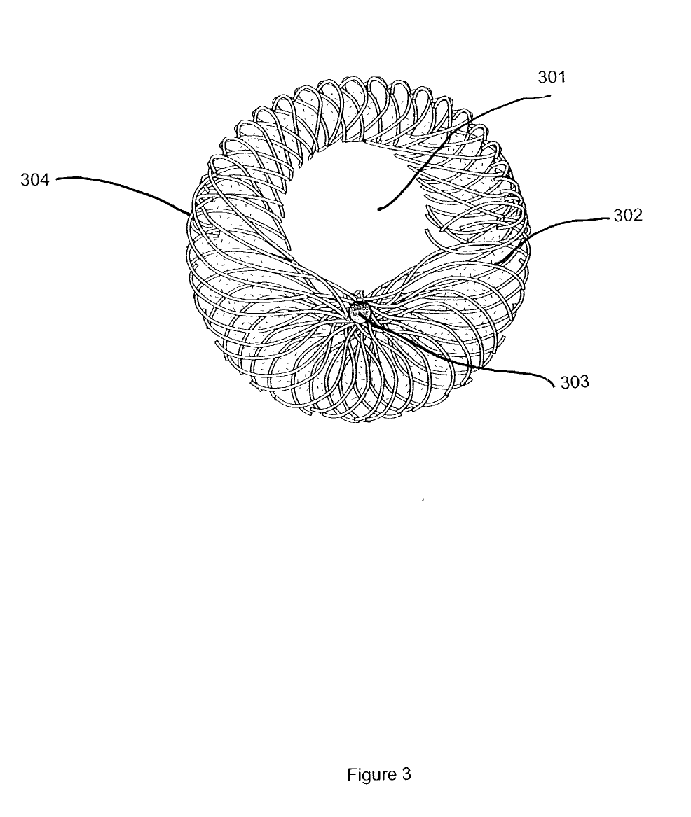

FIG. 3 illustrates a proximal view of a POD with one circular lumen 301. The mesh 302 inhabits the space from the single circular lumen 301 to the outer circumference of the POD 304. The catheter attachment port 303 is eccentrically located.

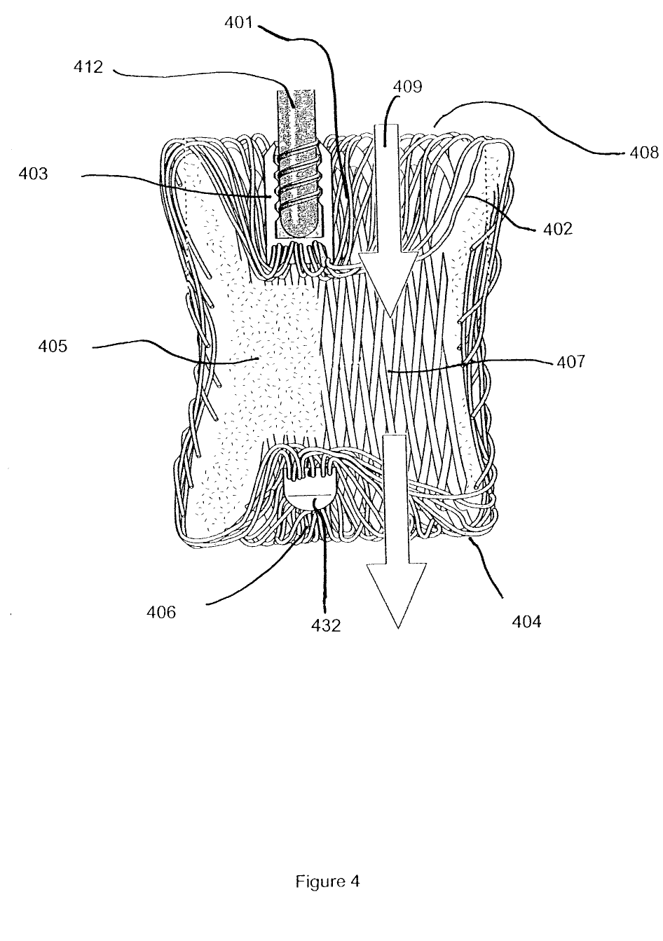

FIG. 4 illustrates a cross-sectional side view of a tapered POD. The blood flow 409 enters through the proximal end 408 and exits through the distal end 406. The area between the proximal 408 and distal end 406 is the center portion 407. Again, the mesh 402 inhabits the space from the lumen 401 to the outer circumference of the POD 404. A fibrous substrate 405 fills the mesh 402 interior. The purpose of the substrate in this embodiment is to further occlude the flow of blood. In an alternative embodiment, the mesh 402 may be without a fibrous substrate 405. This POD is tapered in that the proximal end 408 where the blood flow 409 enters is greater in diameter than the distal end 406. The lumen 401 is considerably smaller in diameter than both the proximal end 408 and the distal end 406. The catheter 412 is to be removed from the catheter attachment port 403 after deployment. The catheter attachment point end 432 may be used as a marker to identify the location or orientation of the POD.

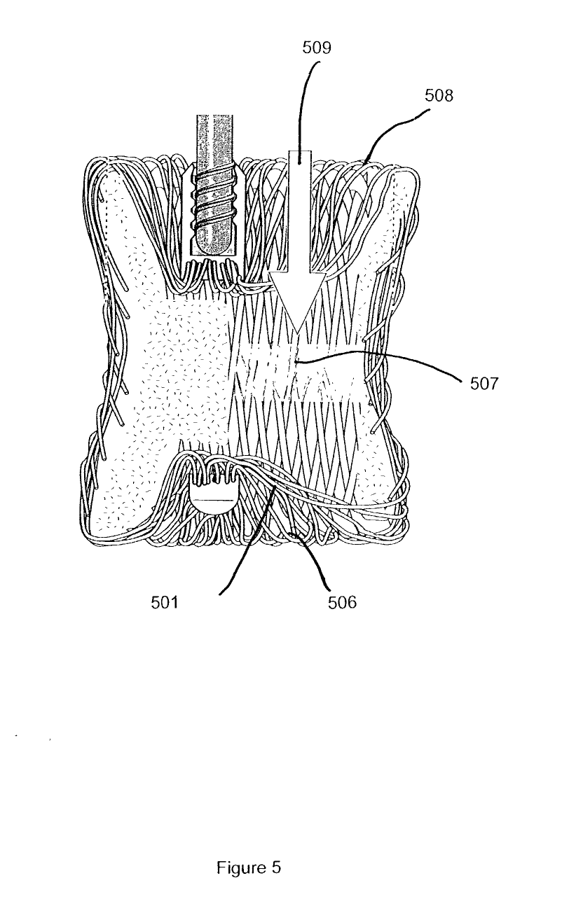

FIG. 5 illustrates a cylindrical or non-tapered POD, such that the proximal end 508 has a diameter that is equal to that of the distal end 506. The blood flow 509 enters through the proximal end 508, flows through the center portion 507 and exits through the distal end 506. Again, the lumen 501 is considerably smaller in diameter than both the proximal end 508 and the distal end 506.

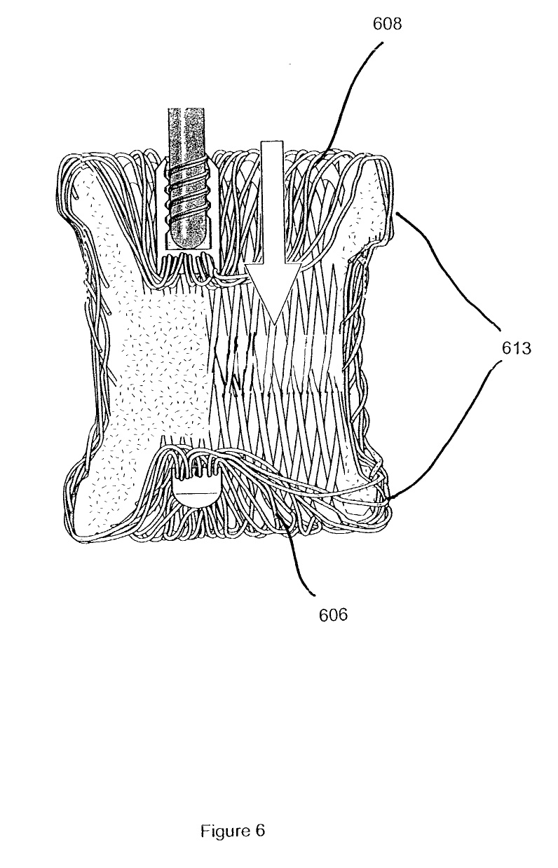

FIG. 6 illustrates a cylindrical or non-tapered POD with a lip 613. In this embodiment both the proximal end 608 and the distal end 606 are of the same diameter and have a lip 613.

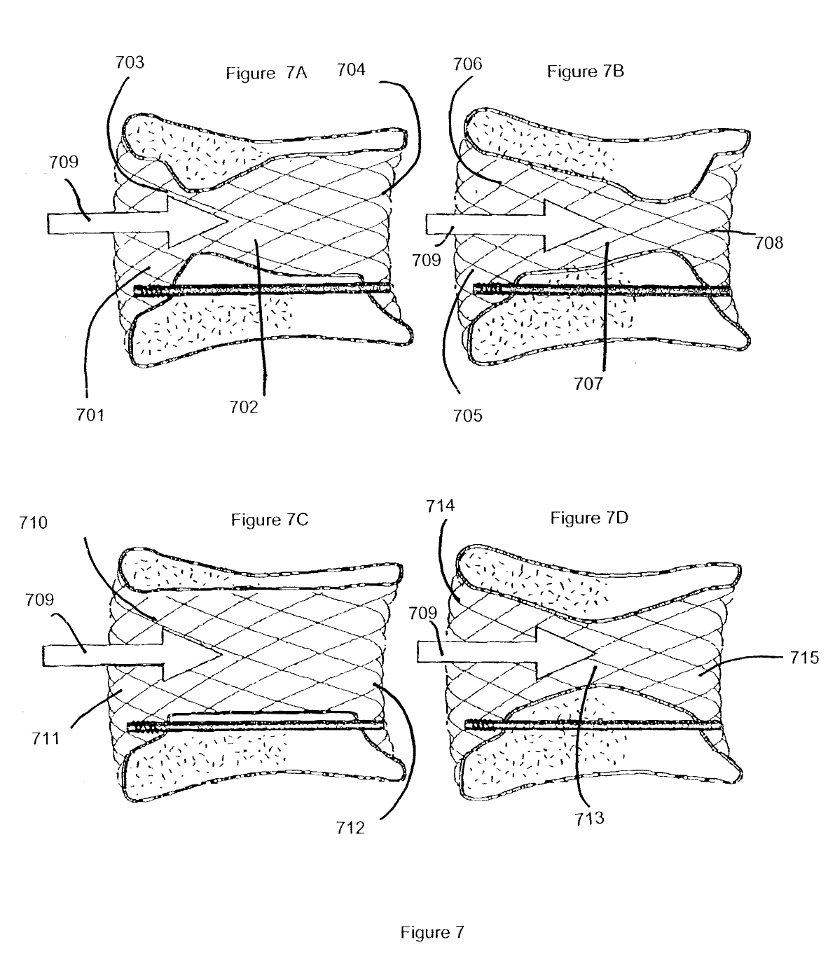

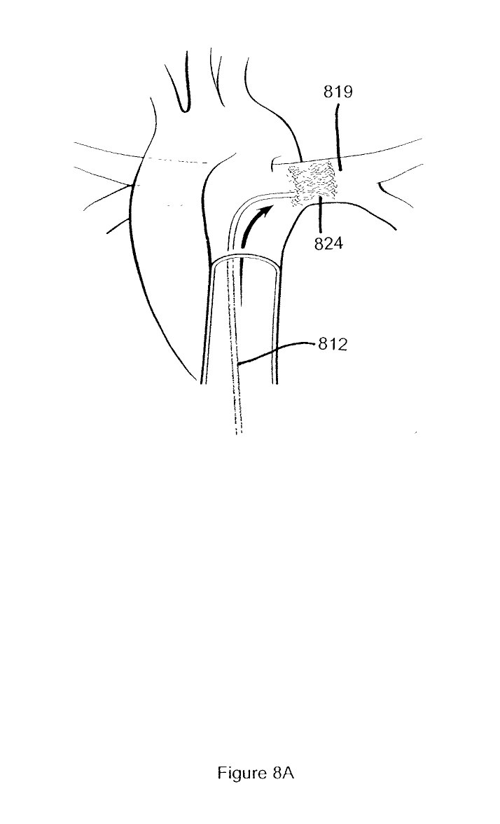

FIGS. 7A through 7D illustrate alternative embodiments of internal configurations for a POD where the lumen is shaped in a particular way to create a particular geometry for handling matter flow there through. FIG. 7A illustrates a cross-sectional side view of an inverted funnel POD configuration, where the blood flow 709 meets a proximal end 701 that narrows toward the center portion 702, the lumen 703 widens immediately through the center portion 702 and the blood flow 709 exits through a wide distal end 704. FIG. 7B illustrates a cross-sectional side view of a funnel POD configuration where the blood flow 709 enters through a wide proximal end 705. The lumen 706 narrows past the center portion 707 and the blood flow 709 exits through a slightly widened distal end 708. FIG. 7C illustrates a cross-sectional side view of a cylinder POD where the internal diameter of the lumen 710 remains relatively constant throughout the length of the POD. The proximal end 711 and the distal end 712 each have a slight widening. FIG. 7D illustrates a cross-sectional side view of a central constriction POD where the central portion of the lumen 713 is narrowed, and each of the proximal 714 and distal ends 715 are slightly widened.

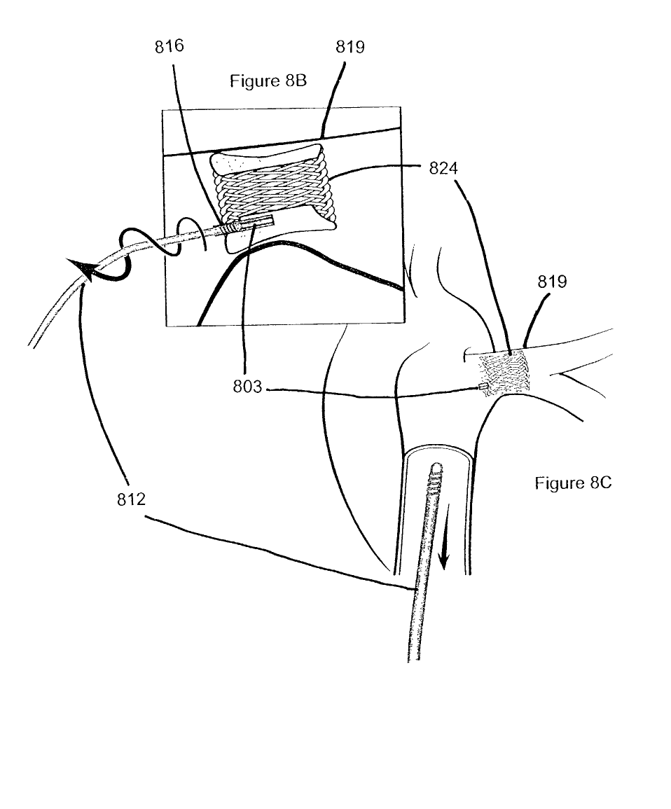

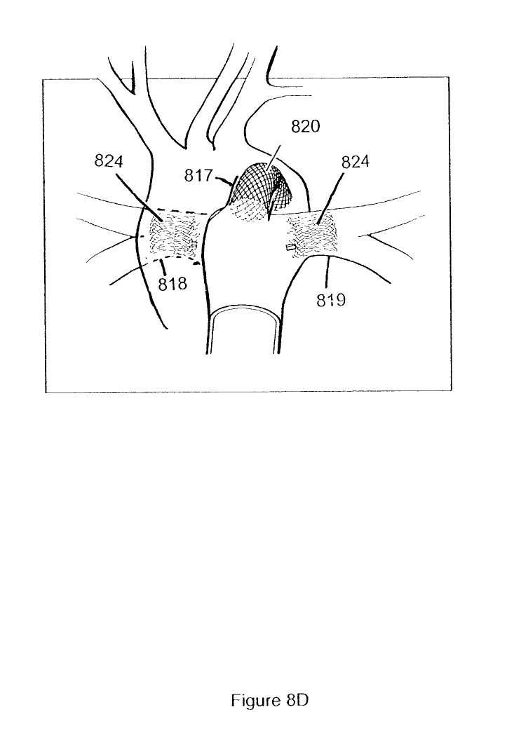

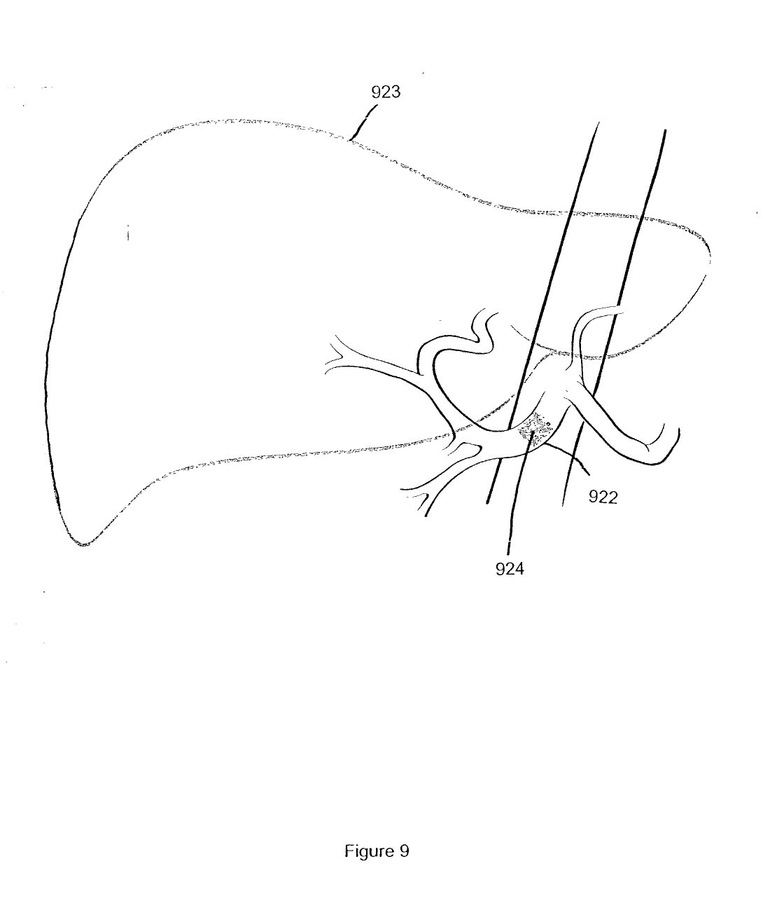

The representations on FIGS. 8A through 8D are depicted in anatomical orientation, where the designations left and right refer to the left and right sides of the depicted individual. For example, in relation to a heart, left is the left side of the individual's heart, depicted on the right side of the page.

FIGS. 8A through 8D demonstrate an embodiment of placement of a POD in the pulmonary arteries. FIG. 8A demonstrates a POD 824 threaded upon a catheter 812 and placed in the desired position in the left pulmonary artery 819. FIG. 8B demonstrates the release of a POD 824 from a catheter 812 upon placement in the desired position in the left pulmonary artery 819. The catheter 812 and the catheter attachment port 803 have matching threads forming an engagement mechanism 816, however one of skill in the art will appreciate alternative methods of engagement are available. FIG. 8C demonstrates the withdrawal of a catheter 812 from the POD 824 after being located within the left pulmonary artery 819. FIG. 8D illustrates the placement of PODs 824 in the right 818 and left pulmonary arteries 819 and a stent 820 within the pulmonary ductus arteriosus (or ductus arteriosus) 817 to treat HLHS. The method of placement of PODs in FIG. 8D may be similar to that shown in FIGS. 8A through 8C, or one of skill in the art may employ different methods.

FIG. 9 illustrates the placement of a POD 924 within the hepatic artery 922 to treat hepatic disorders. FIG. 9 is intended to illustrate that a POD may be used to decrease or regulate flow through any vessel, including vessels associated with the liver 923.

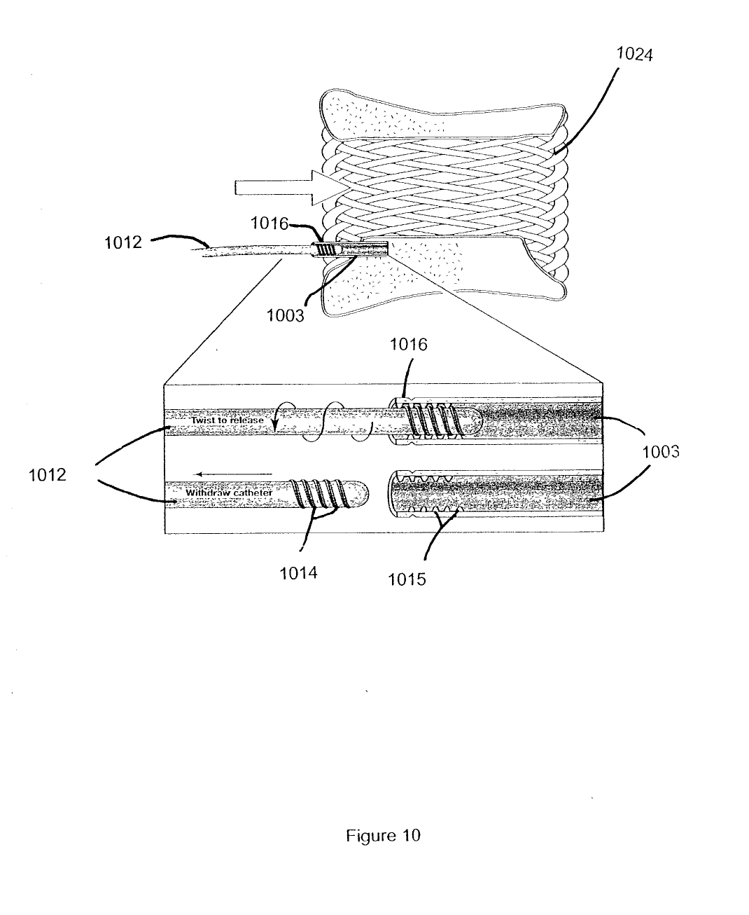

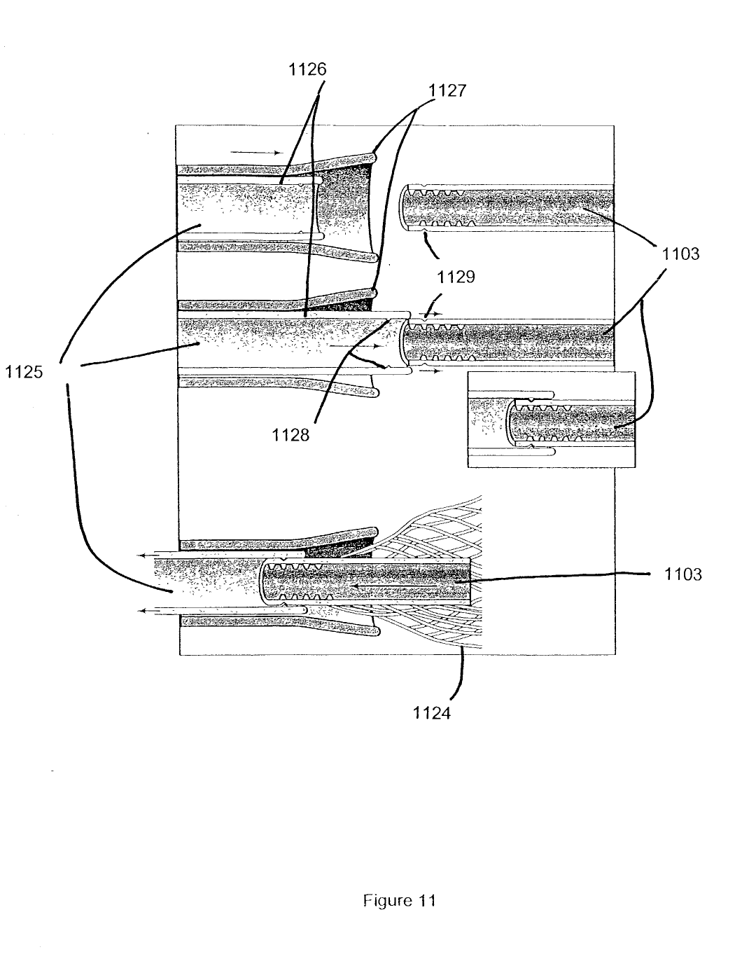

FIG. 10 illustrates a close up view of a catheter attachment port as expanded from a view of the catheter attachment port in relation to the POD. The catheter 1012 can be released from the POD 1024 by disengaging the engagement mechanism 1016 from the catheter attachment port 1003, such that the catheter 1012 has catheter threads 1014 that mechanically engage with the catheter attachment port threads 1015. Alternative engagement mechanisms are possible. Additionally, the catheter 1012 may be reengaged with the catheter attachment port 1003 to act as a retrieval device. One of skill in the art will appreciate that alternative methods of retrieval are also possible.

FIG. 11 demonstrates an embodiment of a retrieval device engaging the catheter attachment port where a clip is used to engage the catheter attachment port to withdraw the POD. The catheter attachment port 1103 is acting as a receptor for the retrieval device 1125. The retrieval device 1125 could be essentially a catheter that allows a clip 1126 to slide through the retrieval device 1125. The retrieval device 1125 depicted has a beveled end 1127 that is slightly larger than the catheter attachment port 1103. The clip 1126 can be slid onto the catheter attachment port 1103. The clip 1126 engages the retrieval device 1125 to the catheter attachment port by sliding the ridge 1128 on the clip 1126 into the notch 1129 on the catheter attachment port 1103. The entire device can then be withdrawn. The ridge 1128 may run along the entire internal circumference of the clip 1126. Alternatively, the ridge 1128 may be less than continuous along the internal circumference of the clip 1126. A notch 1129 runs continuously along the external circumference of the catheter attachment port 1103. Again, the notch 1129 may be less than continuous. The POD 1124 will collapse when a pulling force is exerted on it, thereby avoiding completely occluding the matter transport pathway during retrieval.

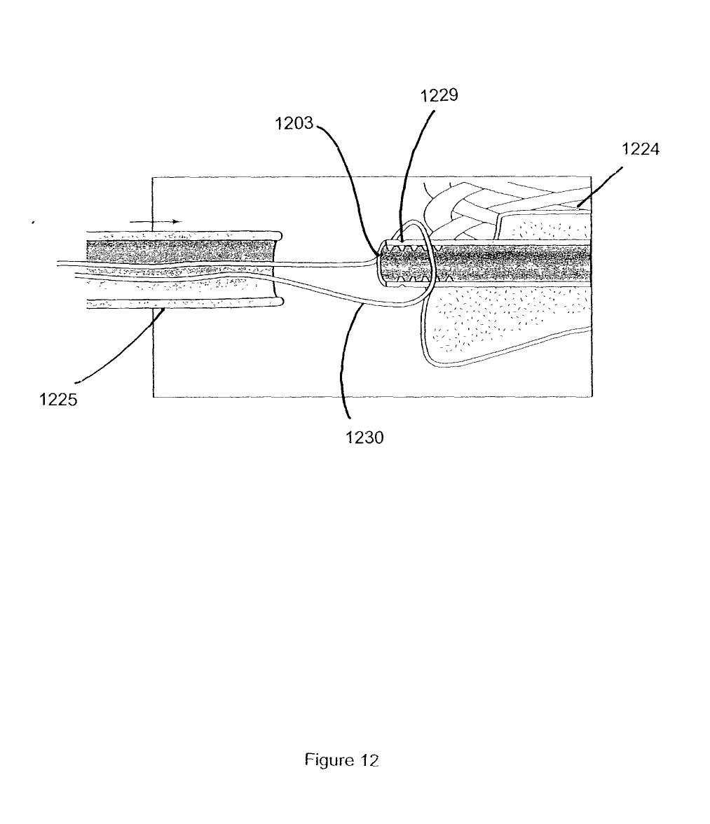

FIG. 12 illustrates an embodiment of an alternative retrieval device where a loop is threaded through the retrieval device 1225 to snare the catheter attachment port and remove the POD 1224. The loop 1230 used to snare the catheter attachment port 1203 engages with a notch 1229 on the catheter attachment port. The loop 1230 is wrapped around the catheter attachment port 1203 and is then cinched tight. The POD 1224 will collapse, as the entire assembly is then withdrawn.

The lumen(s) of the individual PODs may be formed by weaving the mesh such that a hole is made in the proximal end that aligns with a hole in the distal end of the POD. Formation of the lumen(s) in this manner allows for a POD with an interior portion that is hollow, or void of mesh. Alternatively, the mesh may fill the interior, or portions of the interior of the POD, such that the lumen is a continuous tube of mesh extending through the length of the POD. The lumen(s), as viewed from the proximal end of the POD, can be square, rectangular, n-sided polygon (where n is an integer), triangular, circular, two discrete circles, oval, etc. Each of these shapes can be used with the internal configurations of an inverse funnel, funnel, cylinder, central constriction, etc.

The mesh contributes to the dimensions and form of the POD. In a preferred embodiment the mesh is made of nitinol (Nickel Titanium Naval Ordinance Laboratory) wire, as it has shape memory. However, any material with shape memory may be used. Shape memory alloys, such as nitinol, may have thermoelastic martensitic phase transformations that impart shape memory, superelasticity, and high damping capability. Materials without shape memory can also be used. The mesh may be designed such that it helps form the shape and structure of the device, but when being transported it will collapse into a line or less structured shape. The mesh is woven such that when the POD is in place (after transportation) it assumes a structured form, for example, as shown in FIGS. 8A through 9. The structured forms in these figures are representative and not limiting on the variety of structural forms.

When the POD is transported with a catheter or other device the stress of transport deforms the POD. The POD may assume a collapsed form that is thin enough to be moved through blood vessels. Additionally, the ability to assume a structured, deformed or collapsed shape is a beneficial embodiment that allows for easy removal of the POD from the treatment site, when it is no longer necessary or its utility has lessened. For example, when PODs are used in the treatment of HLHS, their necessity is diminished upon the infant's receipt of a heart transplant. Therefore, after the heart transplant, the PODs can be removed utilizing the ability of the PODs to assume a deformed or collapsed shape. In an alternative embodiment the PODs can be placed at the treatment site or removed surgically.

The use of mesh rather than a solid plug also helps in the healing of the placement site after removal. Scar tissue formation around a solid plug of material would be significant and cause difficulty in removal of a plug. This problem is alleviated with the use of mesh because the scar tissue forms around smaller pieces of material so the removal causes less damage as a whole. This also aids in the simple dislodging of the device.

The interior of the POD may be filled with a fibrous substrate, or be vacant. In an embodiment the fibrous substrate is included as a deposition substrate for substances and cells (described below as biological materials and cells, but many kinds of substances are contemplated) from the patient. The deposition of these materials allows for greater occlusion. For example, in an embodiment where PODs are used to treat HLHS, the fibrous substrate in the PODs may be the site of deposition of substances and cells from the infant. The deposition of the substances and cells would further occlude the pulmonary artery(ies) such that blood flow to the lungs would be further decreased. In this manner further protection of the pulmonary bed from pulmonary hypertension may be achieved.

Alternatively, the fibrous substrate may be used for delivery of agents to the treatment site, such that the POD can act as a self-contained unit providing continual or limited delivery of agents. When the fibrous substrate is used it can be located anywhere inside or outside the POD. The fibrous substrate may be at the proximal end, the distal end, throughout the interior of the mesh, or any variation of placement. Additionally, the fibrous substrate can be anchored in place by a variety of methods including, but not limited to, weaving it into the mesh.

As an example, the dimensions of a tapered POD may be as follows: the proximal end with a diameter of 7 mm, the distal end with a diameter of 6 mm, and the central portion with a diameter of 5-6 mm. The lumen(s) of such a POD may have a 3 mm diameter. This POD is referred to as a 7 mm tapered POD as indicated by the diameter of the proximal end. However, the POD may also be a 5 mm tapered POD, a 9 mm tapered POD or an 11 mm tapered POD. Diameters other than those discussed here are also possible. In addition, the other dimensions of the POD can be changed proportionally to the proximal end diameter so that the overall relationships of size between all of the points remains the same, or the other dimensions can be changed disproportionately to create a new structure that is adapted to a particular need. Although specific dimensions are utilized in this and other descriptions the actual dimensions may vary to optimize the results.

The shape and size of the lumen and overall device may be varied to optimize performance. The POD dimensions can be varied to achieve an ideal POD size to vessel size ratio. This ratio was determined in one embodiment by angiographic patency of the POD with pressure gradients of 21-35 mmHg. In this embodiment the PODs were used in newborn lambs to decrease the pressure on the pulmonary bed while the ductus arteriosus was maintained with a stent. The PODs ranged from 7.5 to 14.5 mm in external diameter and the vessels ranged from 5.5 to 11.5 mm in internal diameter. The ideal POD size to vessel size ratio that maintained patency and the correct pressure gradient, was found to be 1.55. Drug and cell delivery may require a different ratio.

An alternative embodiment of a POD is a cylindrical or non-tapered POD where the proximal and distal diameters are the same. A lip may exist on the proximal and/or distal ends of the POD. The lip is not limited to the cylindrical POD, it may also be on any form of the POD. The lip may aid in securing the POD.

Different POD configurations are possible by manipulating the internal shape of the lumen. An inverted funnel POD configuration allows the blood flow to meet a narrow portion of the POD near the proximal end, continue through a widened center portion, and then exit through a widened distal end. A funnel POD configuration allows the blood flow to meet a proximal end that is wider than the center portion. The lumen narrows past the center portion before exiting through a slightly widened distal end. A cylindrical POD configuration has an internal diameter that remains constant throughout most of the length of the POD with a slight widening at the proximal and distal ends. A central constriction POD configuration provides a constriction or narrowing at or near the center portion of the POD.

The internal configuration of the POD may be different than the external structure. Any combination of external structures and internal configurations is possible. Additionally, external structures and internal configurations not illustrated here are possible. The structures and configurations must be adapted to the particular needs for which the POD is used.

In a preferred embodiment the PODs are placed in the pulmonary arteries to decrease the pressure on the pulmonary bed while maintaining the patency of the ductus arteriosus with a stent to treat HLHS. The POD may be guided upon a catheter to the desired location, however alternative methods of placement are available. The POD is released from the catheter via disengaging matching threads on the catheter and the catheter attachment port. However, one of skill in the art may use alternative methods of engagement/disengagement. A POD may also be placed within the hepatic artery of the liver, for treatment of hepatic disorders. Additionally, a POD may be used to decrease or regulate flow through any particular vessel.

Modern medicine is advanced through targeted treatment of areas of the body rather than systemic treatment. A POD can be used to affect targeted treatment by acting as a self-contained unit providing continual or limited delivery of an agent or cells. In one embodiment, a POD is used to deliver an agent, such as insulin. The POD material may be soaked in the agent, or the POD may contain or otherwise carry the agent, such that the sites downstream of the POD are subjected to the agent but not those upstream. Alternatively, the fibrous substrate within the POD may carry the agent for release at the treatment site. Additionally, the amount of agent delivered by the POD can be calibrated so that its dilution at a certain point downstream is known. In this way the effective zone of treatment by the agent can be localized at a particular site.

In another embodiment the POD can be used to deliver cells or other biological materials to a treatment site. The cells or biological materials may be carried in the mesh or the fibrous substrate of the POD. The use of the POD in this embodiment may be targeted treatment of a site through delivery of cells or biological materials with properties that are useful in treating disorders or injuries at or near the treatment site. In another embodiment substances, including biological material, or cells can be deposited upon the POD passively and thereby cause greater occlusion. Alternatively, scavenging and concentration of useful substances, including biological materials, or cells at the treatment site may occur allowing targeted passive treatment at the treatment site.

In another embodiment a material for the POD may be selected such that it scavenges an agent, effectively diminishing the concentration of or removing the agent. In this manner sites downstream of the POD are not subjected to the agent or at least may be protected from higher concentrations of the agent. Additional ways of affecting flow of material or delivery of agents using PODs are possible. For example, the placement of pancreatic eyelet cells can provide delivery of insulin into the blood stream.

The catheter attachment port may be anchored in the mesh and can be located centrally or eccentrically. The catheter attachment port is useful in both the placement and removal of the POD. In one particular embodiment of a POD, the catheter and catheter attachment port have matching threads that are engaged similarly to that of a nut and bolt. This mechanical engagement is only one of many ways of engaging the catheter and the catheter attachment port. As an example, the catheter may be stainless steel and the catheter attachment port may be brass. However, the use of any biocompatible material, such as metal, alloys, titanium, composites, ceramics, plastics, synthetics, is available. In addition, the catheter attachment point end may serve as a marker for location or orientation of the POD.

One skilled in the art will recognize that there are many ways to engage a catheter to a device. Those ways of engaging a catheter to a device are envisioned as different embodiments of the inventions. In a preferred embodiment, the POD retrieval device engages with the catheter attachment port. However, the retrieval device may utilize a different type of engagement, such as using the same structure (catheter attachment port) but a different engagement mechanism, or an entirely different structure than that of the catheter attachment port, any design that offers controlled release and retrievability is available.

A retrieval device may utilize a clip or a loop/snare to engage the catheter attachment port, or some other structure. Additionally, a pin and sleeve coupling, overlapping knuckle or hook and eye attachment is also possible. The retrieval device may be an identical catheter to the one used to deploy the POD, the same type of catheter but with a different engagement mechanism, or a different kind of catheter with a similar or different engagement mechanism. It should be appreciated by those in the art, upon understanding of the inventions, that many different methods of engaging a catheter and a device are possible and all of those methods are part of the inventions.

Additionally, placement and retrieval of the POD may be done through the use of an external imaging modality, such as fluoroscopy or magnetic resonance imaging (MRI). Fluoroscopy, for example, allows one to track the location and orientation of the POD by viewing a marker, such as the catheter attachment port end.

Most of the above discussion involved placement of the POD in a blood vessel. It should be appreciated that a POD or multiple PODs (in series or parallel) could be used in a variety of fluid transport pathways, including living and non-living pathways. Also, other types of flowing matter transport pathways can be partially occluded with PODs. A matter transport pathway may be a blood vessel, such as an artery or vein. A matter transport pathway may be anything that can contain or direct flow of matter, or contain and direct matter. The matter transported may be blood.

The present inventions may be embodied in other specific forms without departing from its spirit or essential characteristics. The described embodiments are to be considered in all respects only as illustrations and not restrictive on the present inventions. Obvious modifications or variations are possible and foreseeable in light of the above teachings. These embodiments of the inventions were chosen and described to provide the best illustration of the principles of the inventions and its practical application to thereby enable one of ordinary skill in the art to make and use the inventions, without undue experimentation. The scope of the inventions is, therefore, indicated by the appended claims rather than by the foregoing description. All changes that come within the meaning and range of equivalency of the claims are to be embraced within the scope of the claims. Distinguishing remarks made within the foregoing description are not to be construed as in any way limiting the claims to less than what is written within the claims themselves. Unless otherwise expressly stated by applicant or applicant's agent, amendments made to any part of this application, and subsequent applications that depend on this application for priority, during prosecution are not done for any reason related to patentability.

I claim:

1. A device for partially occluding a matter transport pathway of a patient, the device comprising:
(a) a body,
said body including a proximal end, a center portion, and a distal end,
said body being formed at least in part from a mesh material;
(b) at least one lumen,
said lumen permitting matter transported by the matter transport pathway to pass there through,
said lumen being defined by said mesh material,
said lumen extending from said proximal end through said distal end; and
(c) a catheter attachment port,
said catheter attachment port being anchored in said body.

2. A device according to claim 1 wherein said body further includes a fibrous substrate.

3. A device according to claim 1 wherein said mesh material has shape memory.

4. A device according to claim 3 wherein said mesh material is nitinol.

5. A device according to claim 2 wherein said fibrous substrate occupies the entire cross-sectional area of said body except for the area occupied by said lumen.

6. A device according to claim 1 wherein said body is tapered such that said proximal end has a diameter greater than said distal end, and both said proximal end and said distal end have a diameter greater than said central portion.

7. A device according to claim 1 wherein the exterior of said body is cylindrical in shape such that said proximal end and said distal end have approximately the same outside diameter.

8. A device according to claim 7 wherein said body is generally cylindrical in shape and said central portion has an exterior diameter greater than the exterior diameter of said proximal end or said distal end.

9. A device according to claim 7 wherein said body is generally cylindrical in shape and said central portion has a diameter less than the diameter of said proximal end or said distal end.

10. A device according to claim 1 wherein said body comprises a lip on at least one of said proximal and said distal ends.

11. A device according to claim 1 wherein said lumen is triangular in cross-section.

12. A device according to claim 1 wherein said lumen is circular in cross-section.

13. A device according to claim 1 wherein said lumen is located centrally as viewed from either said proximal end or said distal end of the device.

14. A device according to claim 1 wherein said lumen is located eccentrically as viewed from either said proximal end or said distal end of the device.

15. A method for partially occluding a matter transport pathway of a patient suffering from a medical condition, the method comprising the steps of:
a) determining that a patient may benefit from a medical treatment involving use of a partial occlusion device;
b) selecting a matter transport pathway in which to place a partial occlusion device; and
c) locating a partial occlusion device in said matter transport pathway.

16. A method as recited in claim 15 wherein said partial occlusion device has a structure that includes:
(i) a body,
said body including a proximal end, a center portion, and a distal end,
said body further is at least partially constructed from a mesh material;
(ii) a lumen,
said lumen being shaped and sized to permit flow of matter there through,
said lumen extending from said proximal end of said body through said distal end of said body,
said lumen being defined at least in part by said mesh material; and
(iii) a catheter attachment port,
said catheter attachment port being anchored in said body.

17. A method as recited in claim 16 further comprising the step of:
(d) inserting a retrieval device into said matter transport pathway;
(e) causing said partial occlusion device to collapse by exerting a pulling force on it so that said partial occlusion device may be removed from said matter transport pathway without completely occluding said matter transport pathway; and
(f) removing said partial occlusion device from said matter transport pathway.

18. A device for partially occluding blood vessels to decrease blood pressure or decreasing volume of blood flow, comprising:
(a) a body,
said body including a proximal end, a center portion, and a distal end,
said body being constructed at least partially by a mesh material;
(b) at least one lumen for permitting blood flow there through,
said lumen extending from said proximal end through said distal end,
wherein said mesh material defines the periphery of said lumen; and
(c) a catheter attachment port,
said catheter attachment port being anchored in said body.
